(19) United States
(12) Patent Application Publication
(10) Pub. No.: US 2006/0234959 A1
(43) Pub. Date: Oct. 19, 2006

(54) Photodynamic therapy utilizing multiple duty cycle light modulation

(75) Inventors: Merrill A. Biel, Mendota Heights, MN (US); Chester E. Sievert JR., Mahtomedi, MN (US); Mark L. Peterson, Chanhassen, MN (US)

Correspondence Address: FULBRIGHT & JAWORSKI L.L.P., 80 SOUTH EIGHTH STREET, SUITE 2100, MINNEAPOLIS, MN, 55402, US

(73) Assignee: Advanced Photodynamic Technologies, Inc.

(21) Appl. No.: 11/107,105

(22) Filed: Apr. 14, 2005

(51) Int. Cl. A61K 31/7048; A61K 31/14; A61H 33/00

(52) U.S. Cl. 514/28; 607/86; 514/643

(57) ABSTRACT

A method of photodynamic disruption of target cells within a target cell site wherein the target cells are associated with one or more of a sterilization procedure, a biofilm eradication procedure, a sterilization procedure of a medical prosthesis, a treatment of an infection at a tissue site, eradication of cancer cells and a fluid or food decontamination process. Surface acting agents such as benzalkonium chloride or polymyxin B sulfate or both may be utilized. A controllable light source capable of delivering light at a plurality of duty cycles is utilized to cause photoactivation of the photosensitive material resulting in degradation of target cells within the site.

### FIELD OF THE INVENTION

This invention generally relates to the field of light delivery to a target cell site having a therapeutically effective amount of a photosensitizing agent. The invention further relates to modulation of light delivered to the target cell site, and more specifically to a photodynamic therapy utilizing a light source operating at multiple duty cycles.

### BACKGROUND OF THE INVENTION

One form of energy activated therapy for destroying abnormal or diseased tissue is photodynamic therapy (PDT). In general terms, PDT is a two-step treatment process for the treatment of a wide variety of different cancers and diseased tissue. The first step in this therapy is carried out by administering a photosensitive material systemically by ingestion or injection, or topically applying the compound to a specific treatment site on a patient's body, followed by illumination of the treatment site with light having a wavelength or waveband corresponding to a characteristic absorption waveband of the photosensitizer. The light activates the photosensitizing compound, causing singlet oxygen radicals and other reactive species to be generated, leading to a number of biological effects that destroy the abnormal or diseased tissue, which has absorbed or associated with the photosensitizing compound. The depth and volume of the cytotoxic effect on the abnormal tissue, such as a cancerous tumor, depends in part on the depth of the light penetration into the tissue, the photosensitizer concentration and its cellular distribution, and the availability of molecular oxygen, which will depend upon the vasculature system supplying the abnormal tissue or tumor.

Current methods of using PDT as a treatment include injecting a photosensitizer, waiting a sufficient period of time for the photosensitizer to reach its target, and then exposing the target region to light. With currently available PDT regimens for the treatment of disease, one administers the photosensitizer anywhere from about 15 minutes to about 4 days prior to the application of light to allow the photosensitizer time to accumulate in the target disease tissue and to be cleared from normal tissue.

Two concerns in the use of the treatment are safety and effectiveness. There are possible side effects associated with PDT. For example, at the target site, PDT has been associated with the development of inflammation with edema and pain, and even necrosis with scarring. With systemically delivered photosensitizers formulated in either aqueous or organic solvents the side effects can include headaches, nausea, and fever, as well as skin photosensitivity. Moreover, the greater the dosage of photosensitizers used, the greater the risk of these, and potentially other, side effects. However, if too little photosensitizer is used in the treatment, then there is a greater risk of having only a partial response to treatment or recurrence of disease.

Various types of PDT light sources and their methods of use have been described in the prior art literature. Light sources utilizing lasers are usually employed as a light source in administering PDT to shorten the time required for the treatment. However, use of high power lasers may result in photodegradation or “photobleaching” of the photosensitizer.

Although not taught nor suggested by the prior art, it would be preferable to employ a low frequency modulated light source so that tumors and other pathogenic cells, structures and/or target cells can more successfully be treated as well as diffused diseases, including metastasized tumors and other pathological tissue formation resulting from infectious or pathogenic agents, such as bacterial infections or other disease states, such as immunological diseases.

Additionally, a need exists for an improved system for sterilizing medical equipment and for fluid decontamination, such as toxin elimination in air handling equipment and/or liquid handling equipment and food decontamination.

### SUMMARY OF THE INVENTION

In accord with the present invention, a method is defined for administering a photodynamic therapy to a target cell such as target tissue in a mammalian subject. The method includes the step of administering to the subject a therapeutically effective amount of a photosensitizing agent having a characteristic light absorption waveband to the target cell. A surface acting agent can also be administered along with the photosensitizing agent to improve the effectiveness of the therapy. Light having a waveband corresponding at least in part with the characteristic light absorption waveband of the photosensitizing agent is used for irradiating at least a portion of the target cell and/or mammalian subject. A light source used for irradiating the target cell preferably delivers light at two or more duty cycles. Preferably, sufficient time is allowed for the photosensitizing agent preferentially associated with the target cell to “recover” during a low dose rate period of time prior to the step of irradiating with the light at a high dose rate.

The invention may have wide applicability and the target cells may include cancer cells, biofilms, bacteria, viruses, plasmids and prions. As further defined, the target tissue may be selected from the group consisting of: a vascular endothelial tissue, an abnormal vascular wall of a tumor, a solid tumor, a tumor of a head, a tumor of a neck, a tumor of a gastrointestinal tract, a tumor of a liver, a tumor of a breast, a tumor of a prostate, a tumor of a lung, a nonsolid tumor and malignant cells. In yet a further application of the present invention, the target tissue is a lesion or wound.

The invention may also have wide applicability to the treatment of infection sites. A method of treatment may include the steps of identifying an infection site having bacteria and virulence factors related to the bacteria; providing a photosensitive material to the infection site; and modulating the delivery of light from a light source to the infection site to cause bacteria death and detoxification of virulence factors of the bacteria. The virulence factors may include lipopolysaccharide and proteases. The infection site may be an infected burn or wound or bacterial lesion. The infection site may be associated with a periodontal disease.

In yet another application of the invention, the target tissue is bone marrow, or comprises cells afflicted with either an autoimmune disease or an inflammatory disease. A still further application of the present invention, relates to methods for the treatment of diffused disease, where the target tissue may include metastasized tumor cells; immunological cells; tissues infected with pathogenic agents or any other diseased or damaged tissues that are interspersed with normal or healthy tissue.

The methods of the present invention can be used to treat diseases characterized by the presence of vascular and/or neovascular blood vessels and/or hyperproliferative and/or abnormal cells. Examples of such diseases include cancer, in which case the target tissues include tumor vasculature and cancerous and normal cells. Examples of tumors are gastric cancer, enteric cancer, lung cancer, breast cancer, uterine cancer, esophageal cancer, ovarian cancer, pancreatic cancer, pharyngeal cancer, sarcomas, hepatic cancer, cancer of the urinary bladder, cancer of the upper jaw, cancer of the bile duct, cancer of the tongue, cerebral tumor, skin cancer, malignant goiter, prostatic cancer, cancer of the parotid gland, Hodgkin's disease, multiple myeloma, renal cancer, leukemia, and malignant lymphocytoma.

The step of irradiating generally comprises the step of providing a light source that is activated to produce the light. In one preferred embodiment of the invention, the light source is disposed external to an intact skin layer of the mammalian subject during the step of irradiating by transcutaneous irradiation. In another preferred embodiment, the method includes the step of inserting the light source underneath an intact skin layer, where the organ comprises the target tissue, as provided in organ irradiation. In another preferred embodiment, the light source is directed to a body orifice, such as the mouth, for a localized photodynamic therapy.

The photosensitizing agent is preferably selected from the group consisting of methylene blue, toluidine blue, indocyanine green (ICG), aminolevulinic acid (ALA), chlorins, bacteriochlorophylls, phthalocyanines, porphyrins, purpurins, texaphyrins, and other photoreactive agents that have a characteristic light absorption peak in a range of from about 500 nm to about 1000 nm.

The step of irradiating is preferably carried out for a time interval of from about 30 minutes to about 72 hours, or more preferably, from about 60 minutes to about 48 hours, or most preferably, greater than about 2 hours, such as from about 2 hours to about 24 hours, depending upon the photosensitizing or photosensitizer agent used. The step of irradiating the target cell is achieved with a light source operating at two or more duty cycles. Between “ON” periods of irradiating the tissue site, the dose rate of light delivered to the tissue site is substantially decreased for an “OFF” period of time. Preferably the “OFF” period of time is between 90 and 180 seconds, and more preferably greater than about 2 minutes.

The invention is not limited to administering a single photosensitizer dose. Instead, the invention also relates generally to multiple dosing (fractionated dosing) to deliver the photosensitizer in various locations throughout the target tissue, e.g., diseased tissue. One can envision, three, four, five or more separate administrations at various time points prior to, during after the application of activating radiation. The number of administrations of photosensitizer is limited by convenience and comfort to the patient versus the effectiveness of additional doses. The total drug dose is limited by the maximal tolerated dose, which is dependent on the photosensitizer used. By fractionating the drug dose, the same effect may be achieved with a lower drug dose, or a higher therapeutic effect can be achieved with the same drug dose.

Methods according to the present invention may also include the administration of surface acting agents to the tissue site to improve the efficacy of a photodynamic treatment. Surface acting agents, such as the polymixins, colisimethate, and the polyene antifungal agents nystatin and amphotericin, sodium dodecyl sulfate (SDS), and cetrimide, may be utilized in the practice of the present invention. Benzalkonium chloride is another surface acting agent which may be utilized in the present invention.

In yet another application of the invention, the target cells are located on medical equipment subject to sterilization using a method according to the present invention. In other applications, the target cells may be disposed within a fluid handling device, such as an air handling system within a building, motor vehicle, etc. A fluid decontamination system is also envisioned whereby the target cells within a fluid are subject to destruction via methods of the present invention. For example, the present invention may be utilized to decontaminate a water supply in portable or stationary environments. In another application, the present invention may be utilized to decontaminate food or food related products.

Still other representative embodiments and advantages of the present invention and methods of construction of the same will become readily apparent to those skilled in the art from the following detailed description, wherein only the preferred embodiments are shown and described, simply by way of illustration of the best mode contemplated for carrying out the invention. As will be realized, the invention is capable of other and different embodiments and methods of construction, and its several details are capable of modification or adaptation in various respects all without departing from the invention as disclosed and claimed. Accordingly, the appended drawings and description contained herein, as well as the descriptions and drawings contained in the applications and associated documents to which the benefit of priority has been claimed and which are incorporated herein by reference as though fully set forth, are to be regarded as illustrative in nature and not as restrictive or limiting.

### BRIEF DESCRIPTION OF THE DRAWING FIGURES

The foregoing aspects and many of the attendant advantages of this invention will become more readily appreciated as the same becomes better understood by reference to the following detailed description, when taken in conjunction with the accompanying drawings, wherein:

FIG. 1 is a depiction of a patient undergoing a photodynamic therapy.

FIG. 2 is an illustration of a protocol for illuminating a target cell according to the invention.

FIGS. 3 through 6 disclose results from investigations into the effect of modulated light on biofilms at different light does rates.

FIG. 7 is a depiction of a fluid decontamination process for decontaminating an air supply utilizing the present invention.

### DESCRIPTION OF THE PREFERRED EMBODIMENT

This invention is broadly directed to methods for therapeutically treating a target tissue or destroying or impairing a target cell or a biological component by the specific and selective binding of a photosensitizer agent to the target tissue, cell, or biological component. At least a portion of the subject (mammal, medical equipment, sterilizing apparatus, etc.) is irradiated with light at a wavelength or waveband within a characteristic absorption waveband of the photosensitizing agent. The light is administered by a modulated light source providing fractionated light to the target tissue site. It is contemplated that an optimal modulation frequency for the light administered to a patient or target cells will be determined clinically, for example by using a light dose escalation trial and in view of the particular photosensitive materials, surfactants. In a preferred embodiment, operation of the modulated light source defines a pair of duty cycles as further described hereinafter.

The terminology used herein is generally intended to have the art recognized meaning and any differences therefrom as used in the present disclosure, will be apparent to the ordinary skilled artisan. For the sake of clarity, terms may also have a particular meaning, as will be clear from their use in context. For example, “transcutaneous” as used in regard to light irradiation in this specification and in the claims that follow, more specifically herein refers to the passage of light through unbroken tissue. Where the tissue layer is skin or dermis, transcutaneous includes “transdermal” and it will be understood that the light source is external to the outer skin layer. However, the term “transillumination” as used herein refers to the passage of light through a tissue layer. For example, “organ transillumination” refers to light irradiation through the outer surface layer of an organ, e.g., the liver, and it will be apparent that the light source is external to the organ, but internal or implanted within the subject or patient. Similarly and more generally, “interstitial transillumination” refers to light irradiation from a light source that is implanted or surgically positioned underneath the epidermal layer of tissue within an organ, such as the parenchymal or capsular layer of tissue of the organ or tumor mass, where the organ or tumor mass comprises the target tissue.

One aspect of the present invention provides for the administering of photosensitive agents or drugs and compounds to specific targets of a subject or patient and activating the photosensitizer agents by subsequently administering to the subject light at a relatively low frequency rate, over a prolonged period of time, from a light source that is internal or external to the target tissue in order to achieve maximal cytotoxicity of the abnormal tissue, with minimal adverse side effects or collateral normal tissue damage.

As used in this specification and the following claims, the terms “target cells” or “target tissues” refer to those cells or tissues, respectively that are intended to be impaired or destroyed by PDT delivered in accord with the present invention. Target cells or target tissues take up or link with the photosensitizing agent, and, when sufficient light radiation of the waveband corresponding to the characteristic waveband of the photosensitizing agent is applied, these cells tissues are impaired or destroyed. Target cells are cells in target tissue, and the target tissue includes, but is not limited to, vascular endothelial tissue, abnormal vascular walls of tumors, solid tumors such as (but not limited to) tumors of the head and neck, tumors of the gastrointestinal tract, tumors of the liver, tumors of the breast, tumors of the prostate, tumors of the lung, nonsolid tumors and malignant cells of the hematopoietic and lymphoid tissue, other lesions in the vascular system, bone marrow, and tissue or cells related to autoimmune disease.

Further, target cells include virus-containing cells, and parasite-containing cells. Also included among target cells are cells undergoing substantially more rapid division as compared to non-target cells. The term “target cells” also includes, but is not limited to, microorganisms such as bacteria, viruses, fungi, parasites, and infectious agents. Thus, the term “target cell” is not limited to living cells but also includes infectious organic particles such as viruses. As the term is used herein and in the appended claims, “target cells” also includes “target compositions” or “target biological components” including, but are not be limited to: toxins, peptides, polymers, and other compounds that may be selectively and specifically identified as an organic target that is intended to be impaired, irreversibly damaged or destroyed by this treatment method.

Target cells may be located at infection sites, biofilms or oral wounds. In other embodiments of the present invention, target cells may be located upon medical equipment such as endotracheal tubes, endoscopes, etc. In such applications, the present invention may be utilized to sterilize medical equipment by photodynamically destroying target cells. In yet other embodiments of the present invention, the target cells may be located within a fluid handling device such as an air duct in a HVAC system or a water decontamination device. For example, the target cells may be disposed upon an air filter subsequent to removal from an air space during a decontamination process. Target cells may also be located upon food products subject to a decontamination process.

“Non-target cells” are all the cells of a mammal that are not intended to be impaired, damaged, or destroyed by the treatment method rendered in accord with the present invention. These non-target cells include but are not limited to healthy blood cells, and other normal tissue, not otherwise identified to be targeted.

In yet another application of the invention, the target tissue is bone marrow, or comprises cells afflicted with either an autoimmune disease or an inflammatory disease. A still further application of the present invention, relates to methods for the treatment of diffused disease, where the target tissue may include metastasized tumor cells; immunological cells; tissues infected with pathogenic agents or any other diseased or damaged tissues that are interspersed with normal or healthy tissue. “Diffused disease” is used herein to refer to a pathologic condition, wherein impaired or damaged tissue is not localized but found in multiple sites throughout the mammalian subject.

“Destroy” means to kill or irreversibly damage the desired target cell. Target cells are understood to be impaired or destroyed even if the target cells are ultimately disposed of by macrophages.

“Photosensitizing or photosensitizer agent” is a chemical compound that is absorbed by or preferentially associates with one or more types of selected target cells and, when exposed to light of an appropriate waveband, absorbs the light, causing substances to be produced that impair or destroy the target cells. Virtually any chemical compound that preferentially is absorbed or linked to a selected target and absorbs light causing the desired therapy to be effected may be used in this invention. In particular applications, the photosensitizing agent or compound is nontoxic to the animal to which it is administered or is capable of being formulated in a nontoxic composition that can be administered to the animal. Photosensitive agents or compounds include, but are not limited to, chlorins, bacteriochlorins, phthalocyanines, porphyrins, purpurins, merocyanines, psoralens, benzoporphyrin derivatives (BPD), and porfimer sodium and pro-drugs such as deltaaminolevulinic acid, which can produce photosensitive agents such as protoporphyrin IX. Other suitable photosensitive compounds include methylene blue, toluidine blue and any other agent that absorbs light in a range of 500 nm-1100 nm.

The term “preferentially associates” or “preferential association” is used herein to describe the preferential association between a photosensitizing agent and target tissue, such as tumor cells or tumor tissue. More specifically, the present invention provides for the photodynamic therapy of a mammalian subject, where the preferential association by photosensitizing agents for target tissue, including tumor cells or tumor tissues, results in the destruction or damage to target tissue upon irradiation. The surrounding normal or healthy tissue is not damaged, where the photosensitizing agent clears much more rapidly from normal cells or tissues than it does from target tissue.

Photosensitizers useful in the described methods can be prepared or formulated for administration in any medium known to the skilled artisan including, but not limited to, tablet, solution, gel, aerosol, dry powder, biomolecular matrix. Photosensitizers useful in the new methods can be administered to a subject by any means known to the skilled artisan including, but not limited to, oral, systemic injection (e.g., intramuscular, intraperitoneal, subcuticular, venous, arterial, lymphatic etc.), topical delivery, topical delivery by a medium (e.g., slow release formulations via photosensitizer impregnated hydrogel polymers), inhalation delivery (e.g., dry powder, particulates), microspheres or nanospheres, liposomes, erythrocyte shells, implantable delivery devices, local drug delivery catheter, perivascular delivery, pericardial delivery, eluting stent delivery. Photosensitizers can also be conjugated to targeting agents, such as antibodies directed to specific target tissues (e.g., tumor-associated antigens or vascular antigens, such as the ED-B domain) and microorganisms (e.g., bacteria, viruses, fungi, and microbial virulence factors).

Ligands directed against receptors that are up-regulated in tumor cells can also be conjugated to photosensitizers. For example, low-density lipoprotein (LDL) can be conjugated to photosensitizers to be directed at tumor cells that express the LDL receptor, and estrogen can be used to target photosensitizers to estrogen receptor expressing cells, such as found in hormone-dependent tumors. Liposomes and immunoliposomes can also be used as targeting agents to carry the photosensitizers to specific target tissues and microorganisms.

Photosensitizers are used in the disclosed methods in “effective amounts,” i.e., at a dosage that facilitates the desired biological effects, for example blood vessel and/or tissue destruction. A useful dosage of a photosensitizer in the new methods depends, for example, on a variety of properties of the activating light (e.g., wavelength, energy, energy density, intensity), the optical properties of the target tissue, and properties of the photosensitizer. The upper and lower dosage limits depend on the type of photosensitizer used, and these limits are generally known for a variety of photosensitizers. In addition, the photosensitizer dosimetry can be determined empirically by those skilled in the art utilizing the methods shown in the examples. One factor in determining the dosage per administration is the number of administrations to be given prior to light treatment. Thus, in the new methods, the dosage can be lower than typically used with a given photosensitizer so that the total of all fractionated doses can be the same or lower than the standard dose for a given photosensitizer.

“Radiation” as used herein includes all wavelengths and wavebands. Preferably, the radiation wavelength or waveband is selected to correspond with or at least overlap the wave length(s) or wavebands that excite the photosensitive compound. Photosensitive agents or compounds typically have one or more absorption wavebands that excite them to produce the substances, which damage or destroy target tissue, target cells, or target compositions. Even more preferably, the radiation wavelength or waveband matches the excitation wave length or waveband of the photosensitive compound and has low absorption by the non-target cells and the rest of the intact animal, including blood proteins. The radiation used to activate the photosensitive compound is further defined in this invention by its intensity, duration, and timing with respect to dosing a target site. The intensity or dosage rate must be sufficient for the radiation to penetrate skin and reach the target cells, target tissues, or target compositions. The total light dose must be sufficient to photoactivate enough photosensitive agent to achieve the desired effect on the target site. Both intensity and duration are preferably limited to avoid over treating the subject or animal.

The terms “chemical agent” and “surface acting agents” and “surfactants” as used herein are broadly defined to include materials, compounds, agents, chemicals, solutions, or substances which alter the energy relationships at molecular interfaces. Among the manifestations of these altered energy relationships is the lowering of surface or interfacial tensions. Chemical agents or compounds displaying surface activity are characterized by an appropriate structural balance between one or more water-attracting groups and one or more water-repellent groups. Surfactants are characterized by having two different moieties, one polar and the other nonpolar. The polar moeity is referred to as hydrophilic or lipophobic, and the nonpolar as hydrophobic or lipophilic. The electrical charge on the hydrophilic portion of a surface acting agent may serve as a convenient basis of classification of these compounds. Surface active agents have been classified as: Anionic, Cationic, Non-Ionic, and Amphoteric. Other classes of surfactants are also known or may be developed or defined in the future. Chemical agents, such as surfactants, are known to affect the permeability of cell membranes, and membrane-like structures of acellular organisms, such as capsids and envelopes. The ability of these chemical agents or surfactants to become oriented between lipid and protein films is thought to produce a disorientation of the membrane of microorganisms, so that it no longer functions as an effective osmotic barrier. The term “membrane” as used herein is meant to broadly include cellular or acellular organism structures, such as cell walls, cytoplasmic membranes, cell envelopes, coverings, capsids, envelopes, or other types of boundary-defining terms of cellular or acellular organisms. It is believed that a photosensitive material may diffuse through the membrane of a microorganism having a surfactant-compromised membrane. A photosensitive material concentration within the membrane and the organism increases over time via osmotic diffusion of the photosensitive material across the surfactant-compromised membrane. The polymixins, colisimethate, and the polyene antifungal agents nystatin and amphotericin are surfactants, as is sodium dodecyl sulfate (SDS). Cetrimide and benzalkonium chloride are also known surfactants.

A light source is utilized to practice embodiments of the present invention. The light source may be laser light source, a high intensity flash lamp, or other illumination sources as appreciated by those skilled in the relevant arts. A broad spectrum light source may be utilized, however a narrow spectrum light source is one preferred light source. The light source may be selected with reference to the specific photosensitive material, as photosensitive materials may have an associated range of photoactivation. A laser light source may be used to practice the present invention. A variety of laser light sources are currently available, and the selection of a particular laser light source for implementing the PDT would readily be appreciated by those skilled in the relevant arts. A hand manipulable light wand or fiber optic device may be used to illuminate tissue within a living body. Such fiber optic devices may include a disposable fiber optic guide provided in kit form with a solution containing a photosensitive material and a surfactant. Other potential light devices for use in accordance with the present invention include the devices disclosed in applicant's U.S. Pat. No. 6,159,236, entitled Expandable Treatment Device for Photodynamic Therapy and Method of Using Same, and U.S. Pat. No. 6,048,359, entitled Spatial Orientation and Light Sources and Method of Using Same for Medical Diagnosis and Photodynamic Therapy, both incorporated in their entireties by reference herein. The laser source may be selected with regard to the choice of wavelength, beam diameter, exposure time and sensitivity of the cellular and/or acellular organisms to the laser/photosensitizer/surfactant combination. In preferred embodiments, the light source is utilized for a period of time necessary to affect a photodynamic response.

Repeat administrations of a treatment protocol may also be necessary or desired, including repeat administrations of surfactants and photosensitive materials and light activation. The repeat administrations may include different surfactants and/or photosensitive materials than previously administered. Repeat administrations of the treatment protocol may continue for a period of time.

Additional aspects of the present invention include administration or delivery approaches of the photosensitive material and the chemical agent or surfactant. In one example, the photosensitive material and the surfactant are provided in a combined solution and topically applied to the cell site. In alternative embodiments, the photosensitive material may be applied or delivered or dispensed to a tissue site before, during, or after the application or delivery of the surfactant through known delivery/administration approaches. In one preferred embodiment, a topical surfactant application would precede a topical photosensitive material application by 1-30 minutes.

Additional aspects of the present invention further include combinations of different photosensitive materials and different chemical agents or surfactants during a treatment protocol. In one preferred embodiment, a particular combination of a photosensitizer and a surfactant would be dispensed to the tissue site in association with a first photodynamic illumination of the tissue site. After a period of time, another different particular combination of a photosensitizer and a surfactant would be dispensed to the tissue site in association with a second photodynamic illumination of the tissue site.

Yet other aspects of the invention include combining a plurality of different surfactants with a given photosensitive material, and/or combining a plurality of different photosensitive materials with a given surfactant.

In a preferred embodiment, a photosensitive material such as methylene blue or toluidene blue may be used in combination with surfactants, such as SDS, polymyxin B, benzalkonium chloride and/or cetrimide, and activated by light energy to provide broad spectrum antibiotic activity for destroying both gram positive and gram-negative bacteria, funguses, viruses, spores, and/or cancer cells. The photosensitive material and surfactant may be combined in solution and administered to a site to be treated. Solution administration may include topical application, or intravenous, subcutaneous, intratumoral, or peritumoral injection. Additional administration approaches may also be practicable. An intratumoral injection of the solution may be advantageous for photoeradication of tumor cells.

Any radiation source producing a wavelength that can activate the photosensitizer used can be employed in the new methods. In certain embodiments, the radiation source used can be a coherent or a non-coherent source including, but not limited to, a laser, a lamp, a light, an optoelectric magnetic device, a diode, VCSEL device, or a diode laser. The radiation source must be capable of directing radiation to a site of interest, for example, a laser with optical fiber delivery device, or a fiber optic insert, or a lens used for interstitial or open field light delivery, or diffusers, including those that may improve penetration of the radiation through the skin or a node of a tumor.

The intensity of radiation used to treat the target cell or target tissue is preferably between about 5 mW/cm² and about 100 mW/cm². More preferably, the intensity of radiation employed should be between about 10 mW/cm² and about 75 mW/cm². Most preferably, the intensity of radiation is between about 15 mW/cm² and about 50 mW/cm².

The duration of radiation exposure administered to a subject is preferably between about 30 minutes and about 72 hours. More preferably, the duration of radiation exposure is between about 60 minutes and about 48 hours. Most preferably, the duration of radiation exposure is greater than about 2 hours, such as between about 2 hours and about 24 hours.

It is contemplated that a targeted photosensitizer agent can be substantially and selectively photoactivated in the target cells and target tissues within a therapeutically reasonable period of time and without excess toxicity or collateral damage to non-target normal tissues.

One aspect of the present invention is drawn to a method for transcutaneous energy activation therapy applied to destroy tumors in a mammalian subject or patient by first administering to the subject a therapeutically effective amount of a photosensitive material.

The present invention provides a method for providing a medical therapy to an animal, and the term “animal includes, but is not limited to, humans and other mammals. The term “mammals” or “mammalian subject” includes farm animals, such as cows, hogs and sheep, as well as pet or sport animals such as horses, dogs, and cats.

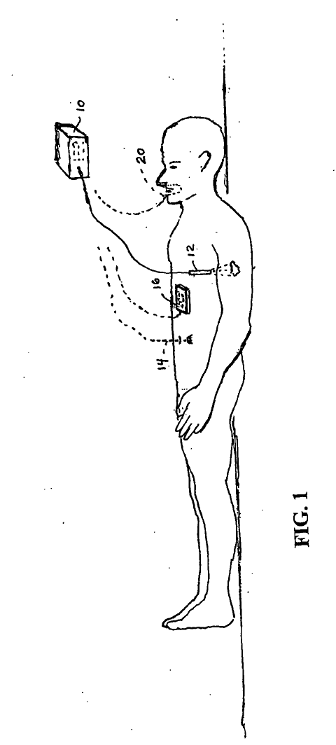

FIG. 1 illustrates the use of an external light source 10 for generating light for illuminating various tissue sites of a patient. Light source 10 can be coupled to a light wand 12 for external illumination of a tissue site. Light source 10 can be coupled to a light device, such as an implantable light probe 14, which penetrates through a dermal layer and into a subcutaneous layer or tumor. Light source 10 can be coupled to a flexible patch 16 containing a hydrogel material such as disclosed in U.S. Ser. No. 11/050,349, entitled Wound Treatment Device for Photodynamic Therapy and Method of Using Same, incorporated by reference herein. Light source 10 can be coupled to a shaped illuminated article, such as a mouth piece 20, for an oral treatment. Other light sources are disclosed in U.S. Ser. No. 10/208,632, entitled Treatment Device for Topical Photodynamic Therapy and Method of Using Same, and incorporated by reference herein. The light from light source 10 activates a photosensitive drug that has been administered to the patient and selectively targeted to cause the drug to destroy the tumor or other pathogenic cells.

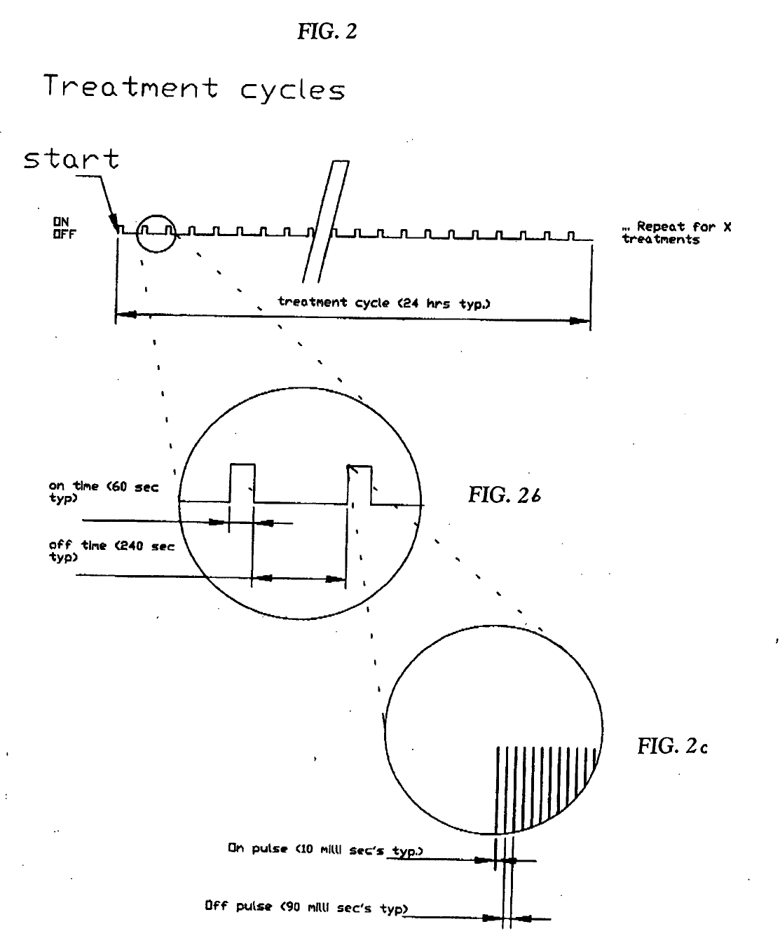

FIG. 2 illustrates one possible illumination protocol for practicing the present invention. FIG. 2 depicts a treatment cycle comprising first and second duty cycles of pulsed light emitted from light source 10. In this example, the treatment cycle extends over a period of time, such as 24 hours. Light source 10 is pulsed at two different duty cycles, including a first duty cycle defining the overall ON/OFF periods of the treatment cycle (FIG. 2b) and a second duty cycle defined by smaller scale ON/OFF periods (FIG. 2c). Each duty cycle is generally defined as the ratio of the sum of all pulse durations to the total period. The term “duty cycle” is defined herein to be the ratio of “ON” durations to the total period of “ON” and “OFF” durations. As illustrated in the example of FIG. 2, the “ON” durations may be further defined by a second duty cycle. That is, the light source 10 may be emitting pulsed light at a second duty cycle while operating in an “ON” mode. See, FIG. 2c.

The treatment cycle can extend from several minutes to several days in length. In the illustrated embodiment, the light source 10 waveform defines a pair of duty cycles which extend over a treatment session of hours. As illustrated in FIG. 2b, light source 10 is activated for 60 second “ON” periods interrupted by “OFF” periods of 240 seconds. In this example, a first duty cycle of 20% is defined by the ratio of 60 seconds to 300 seconds. As illustrated in FIG. 2c, during the ON periods, light source 10 is being pulsed for 10 millisecond “ON” periods interrupted by 90 millisecond “OFF” periods. In this example, a second duty cycle of 10% can therefore be defined by as the ratio of 10 milliseconds to 100 milliseconds.

A treatment session can also include minutes, hours or days of dual-duty cycle pulsed light application separated by periods of non-illumination. A system with more than two pulsed light duty cycles is also practicable. For example, a dual-duty cycle light application as disclosed in FIG. 2 may be utilized regularly for 1 hour periods separated by 1 hour periods of non-illumination. In such an example, a third duty cycle of 50% may be defined. Obviously, the invention may be practiced at other duty cycles, light intensities and light wavelengths as suggested, for example, by experimentation or clinical trials.

FIG. 2 illustrates that light source 10 is activated at uniform “ON” levels, e.g., that the intensity of the light pulses remains constant over time. In other embodiments of the invention, the intensity of the light pulses may assume other configurations, such as ramp, triangular and sawtooth waveforms. The intensity of light emitted from light source 10 during the “ON” periods does not necessarily need to remain constant. Additionally, during an “OFF” period, the intensity of light emitted from light source 10 need not be zero. For example, light source 10 may be operated between an “ON” state and an “OFF” state of a substantially lower (but not zero) light intensity as compared to the “ON” state. Furthermore, during an “OFF” state light source 10 may emit light at a non-constant level. In other words, during the “OFF” intervals, light source 10 may emit light at a substantially lower level and in a non-uniform manner as compared to light emission during an “ON” interval.

In preferred embodiments of the invention, a dark interval associated with the first duty cycle is more than 100 seconds. See, FIG. 2b. The dark interval associated with the first duty cycle is preferably at least twice as long as a light interval associated with the first duty cycle. The first duty cycle is preferably between 10% to 90%. The second duty cycle is preferably between 10⁻⁶% to 90%. More preferably, the second duty cycle is between 10⁻⁶% to 1%. Pulse lengths of light associated with the second duty cycle are preferably less than 0.25 seconds.

In other embodiments of the invention, illumination periods associated with the first duty cycle vary in duration during a treatment session. Additionally, the pulse lengths associated with the second duty cycle can vary in duration during a treatment session.

The photosensitive material may be provided to the target cell in fractionated applications during a treatment session. The step of providing the photosensitive material can occur by providing a solution, powder or paste upon or in proximity to the target cell site. The solution, powder or paste may be provided via one or more of the group containing: a surface application, an injection proximate the target cells, an intravenous injection, a subcutaneous injection, inhalation, a topical application, and a spray or drip application. The step of providing the photosensitive material may be via an impregnation of the photosensitive material on a surface of a medical prosthesis. The step of providing the photosensitive material may be via a release of photosensitive material from a hydrophilic polymer, such as disclosed in U.S. Ser. No. 11/050,349, entitled Wound Treatment Device for Photodynamic Therapy and Method of Using Same, incorporated by reference herein. The photosensitive material may be released from a hydrogel material applied at the target cell site.

In other embodiments of the present invention, the target cells are associated with one or more of a sterilization procedure, a biofilm eradication procedure, a sterilization procedure of a medical prosthesis, a treatment of an infection at a tissue site, eradication of cancer cells, an air decontamination process and a water or food decontamination process. The target cells may typically include microbes, spores, fungi, or cancer cells. The target cells may also include viruses, prions or plasmids.

A method of photodynamic disruption of the present invention may include the step of providing a surface acting agent in association with the target cells. The surface acting agent may be benzalkonium chloride or polymyxin B sulfate or both. Additional details of surface acting agents are disclosed in U.S. patent application Ser. Nos. 10/026,198, 10/052,990, and 10/792,578, each application being incorporated by reference herein. The surface acting agent may contain benzalkonium chloride provided in a concentration range of between 0.001% to 1%. More particularly, the surface acting agent may contain benzalkonium chloride provided in a concentration range of between 0.005% to 0.05%. The surface acting agent may contain polymyxin B sulfate provided in a concentration range of between 1 to 5 μg/ml. The step of providing the surface acting agent may precede the step of providing the photosensitive material by 1 to 60 minutes.

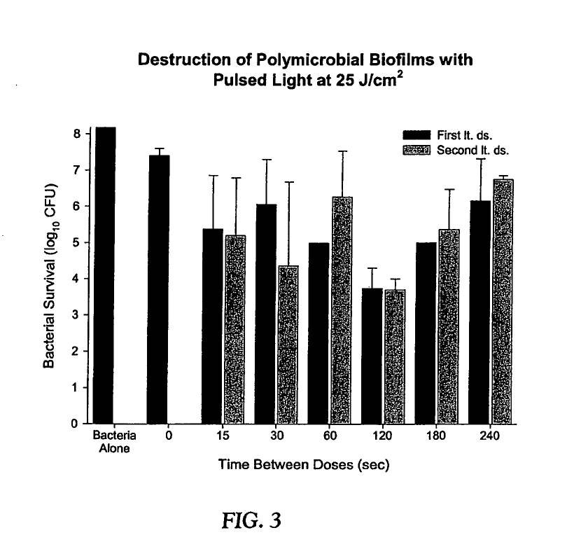

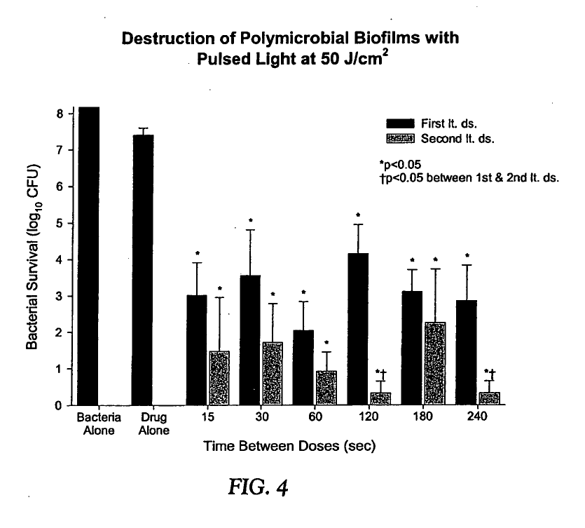

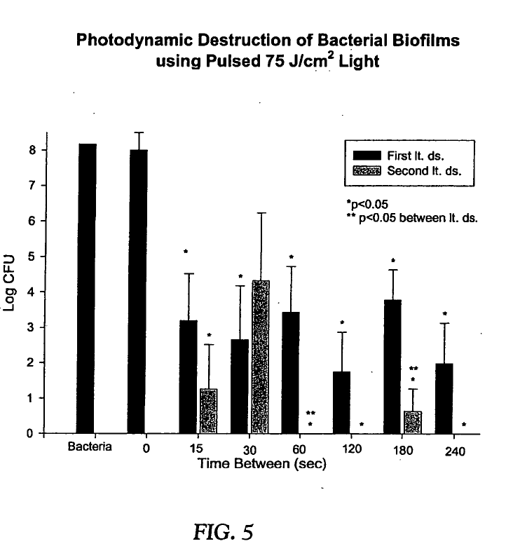

The effect of varying the length of time between light doses on the destruction of bacterial biofilms has been analyzed. FIGS. 3-5 disclose the results of experiments utilizing a method according to the present invention. Bacteria including P. aeruginosa and S. aureus were suspended in 10 mL of trypticase soy broth at a McFarland Standard of 0.5 (1.5×10⁸ cells/mL) as measured by a turbidity nephlometer. One-cm² silicone squares in tissue culture slides were overlaid with 2 mL of the bacterial suspension, and incubated at 37° C. for 24 hours. The photosensitizer solution was from a premade solution of 300 μg/mL of methylene blue, 0.01% of benzalkonium chloride (surface acting agent), and 4 μg/mL of polymyxin. The silicone square surface was covered with the photosensitizer solution using a 1 cc syringe and allowed to sit for 5 minutes before illumination. Each square was exposed to 664 nm light at a dose rate of 100 mW/cm² and two light doses of 25 (FIG. 3), 50 (FIG. 4), and 75 J/cm² (FIG. 5) by a microlens using a spot diameter of 1.35 cm. The time between the light doses was 15, 30, 60, 120, 180, 240 sec. Before applying the photosensitizer solution and after each light treatment the surface was cultured by a swab into 1 mL of saline and serially diluted in saline onto plate count agar and grown overnight at 37° C. Colonies were counted to determine the number of viable bacteria, which will be expressed as CFU. FIGS. 3-5 illustrate the results of the bacterial biolfilm analysis at 25, 50 and 75 J/cm², respectively.

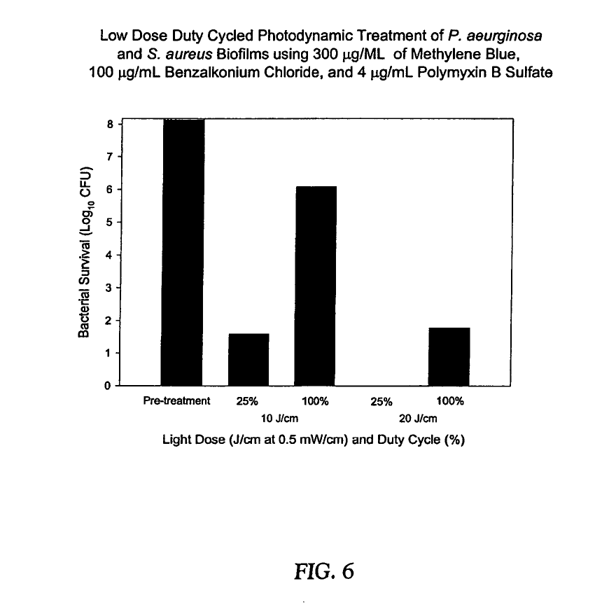

FIG. 6 illustrates the results of another bacterial biofilm analysis using 300 μg/ML methylene blue, 100 μg/ML benzalkonium chloride and 4 μg/ML polymyxin b sulfate. FIG. 6 discloses that duty cycles of less than 100% (continuous light) provide for improved bacterial reduction.

Enhanced microbial destruction has been indicated when the off period is greater than approximately 2 minutes. A photodynamic therapy utilizing a modulated light source providing an “on” period of illumination to the tissue target followed by an off period of greater than about 2 minutes appears to be particularly useful. A range of “off” periods between 90 sec. to 150 sec. may also be utilized to practice the present invention. The “off” period is not necessarily a period without illumination, rather the “off” period is a period with substantially decreased illumination relative to the illumination during the “on” period.

These experiments demonstrated that a pulse duration pause of two or more minutes increases the efficacy of eradication of cells with PDT. This effect is presumed to be due to a combination of allowing the photosensitizer to diffuse into cells that have had their cell membranes partially damaged by the first PDT treatment as well as reducing the photodegradation or photobleaching of the photosensitizer and allowing for reoxygenation of the target cells to occur.

Another embodiment of the present invention is directed to an apparatus for eradicating airborne or fluid biological pathogens using a photosensitizer, a light source, a method for commingling the pathogens with the photosensitizer, and subsequent light exposure of the pathogen at sufficient energy levels at a predetermined wavelength. The photosensitizer may be selected from among a group of photosensitive materials. The light source may be an array of vertical cavity surface-emitting lasers (VCSELs), LEDs, laser diodes, or one or more incandescent bulb(s).

The reduction or elimination of airborne contaminants is desirable, if not essential, in some environments. The increasing threat of the use of biological weapons requires systems for the rapid and complete broad spectrum eradication of pathogens. Although many different gram positive and gram negative bacteria, fungal, or viral pathogens may be employed as biological weapons, the present threats include anthrax, tularemia, plague, Aspergillus, and small pox. The reduction of airborne pathogens in commercial HVAC systems similarly requires rapid and complete broad spectrum eradication of these pathogens.

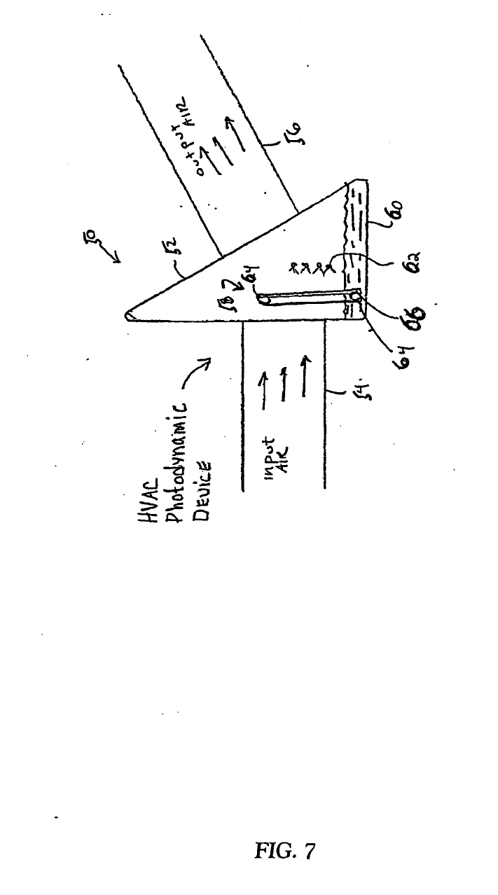

Referring to FIG. 7, fluid filtration device 50 according to the present invention is illustrated. Filtration device 50 may be utilized to sterilize a fluid such as air or water. In one embodiment of the present invention, the device 50 includes an enclosure structure 52 for housing the internal components of the filter 50 and for facilitating transportability of the device 50. The enclosure 52 illustrated may be sized for a building structure and may be placed in-line within the heating-air conditioning-ventilation (HVAC) system of the structure. Airflow through the enclosure 52 enters through an air intake 54 and exits through an air outlet 56. A driven fan or other air motive means (not shown) may be placed in any suitable location within the HVAC system.

Within the enclosure 52 of the device 50 is a filter structure 58, a bath solution 60 including a photosensitive material, optionally pyrrolnitrin, and optionally a surfactant such as SDS, cetrimide, or benzalkonium chloride, and a light source 62 such as a VCSEL array. The filter structure 58 may be a flexible roll structure and may be maintained between rotating rollers 64 which impart a rotating motion to the roll. The lower end 66 of the roll structure may be received into the bath solution 60 which bathes that portion of the filter structure. The filter 58 is positioned to entrap incoming bacteria, fungi, or virus elements within its structure. The entrapped elements are then passed through the bath solution 60 as the filter 58 rotates about the rollers 64. The entrapped elements, then partially or completely enveloped with the bath solution 60 are subjected to illumination from the light source 62 to neutralize the elements.

Filter structures other than the flexible roll structure 58 may be used. For example, a generally flat disc-like filter may be rotated about its axis in a bath solution 60 or even a stationary filter (with photosensitizer being applied upon) may be practicable. The bath solution 20 may be sprayed or otherwise applied to the surface of the filter structure 58 in a variety of known manners which are readily appreciated by those skilled in the art. The filter structure 58 may be replaceable or may be sealed within a disposable enclosure. Sizing of the entire filter 50 can result in the application of the filter technology in a variety of environments, such as building structures, vehicle environments, portable structures, or even in human mask form.

A food decontamination device according to the present invention is also envisioned. Such a device may be utilized to sterilize food or food related products. In one embodiment of the present invention, the food sterilizing process is achieved within an enclosure structure for housing internal components. Food or food-related products may pass through the structure during a decontamination process wherein photosensitive material within or upon the food or food-related products is illuminated with a light source activated in accordance with the present invention. The photosensitive material may be applied to or incorporated into the food or food-related products through known technologies, such as solution spraying, dusting, etc.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

The above described embodiments of the invention are merely descriptive of its principles and are not to be considered limiting. Further modifications of the invention herein disclosed will occur to those skilled in the respective arts and all such modifications are deemed to be within the scope of the invention as defined by the following claims.

1. A method of photodynamic disruption comprising:
identifying a target cell site;
providing a photosensitive material to the target cell site;
providing a controllable light source capable of delivering light at a plurality of duty cycles;
periodically illuminating target cells within the target cell site with light from the light source at a first duty cycle; and
during periods of illumination associated with the first duty cycle, pulsing the light source at a second duty cycle so as to cause photoactivation of the photosensitive material resulting in degradation of target cells within the site.

2. The method of photodynamic disruption of claim 1 wherein a dark interval associated with the first duty cycle is more than 100 seconds.

3. The method of photodynamic disruption of claim 1 wherein a dark interval associated with the first duty cycle is at least twice as long as a light interval associated with the first duty cycle.

4. The method of photodynamic disruption of claim 1 wherein the first duty cycle is between 10% to 90%.

5. The method of photodynamic disruption of claim 1 wherein the second duty cycle is between 10⁻⁶% to 90%.

6. The method of photodynamic disruption of claim 5 wherein the second duty cycle is between 10⁻⁶% to 1%.

7. The method of photodynamic disruption of claim 5 wherein pulse lengths of light associated with the second duty cycle are less than 0.25 seconds.

8. The method of photodynamic disruption of claim 1 wherein illumination periods associated with the first duty cycle vary in duration during a treatment session.

9. The method of photodynamic disruption of claim 1 wherein pulse lengths associated with the second duty cycle vary in duration during a treatment session.

10. The method of photodynamic disruption of claim 1 wherein the photosensitive material is provided to the target cell in fractionated applications during a treatment session.

11. The method of photodynamic disruption of claim 1 wherein the step of providing the photosensitive material occurs by providing a solution, powder or paste upon or in proximity to the target cell site.

12. The method of photodynamic disruption of claim 11 wherein the solution, powder or paste is provided via one or more of the group containing: a surface application, an injection proximate the target cells, an intravenous injection, a subcutaneous injection, inhalation, a topical application, and a spray or drip application.

13. The method of photodynamic disruption of claim 1 wherein the step of providing the photosensitive material is via an impregnation of the photosensitive material on a surface of a medical prosthesis.

14. The method of photodynamic disruption of claim 1 wherein the step of providing the photosensitive material is via a release of photosensitive material from a hydrophilic polymer.

15. The method of photodynamic disruption of claim 14 wherein the photosensitive material is released from a hydrogel material applied at the target cell site.

16. The method of photodynamic disruption of claim 1 wherein the target cells are associated with one or more of a sterilization procedure, a biofilm eradication procedure, a sterilization procedure of a medical prosthesis, a treatment of an infection at a tissue site, eradication of cancer cells and a fluid or food decontamination process.

17. The method of photodynamic disruption of claim 1 wherein the target cells include microbes, spores, fungi, or cancer cells.

18. The method of photodynamic disruption of claim 1 wherein the target cells include viruses, prions or plasmids.

19. The method of photodynamic disruption of claim 1 further comprising the step of providing a surface acting agent in association with the target cells.

20. The method of photodynamic disruption of claim 19 wherein the surface acting agent is benzalkonium chloride or polymyxin B sulfate or both.

21. The method of photodynamic disruption of claim 20 wherein the surface acting agent contains benzalkonium chloride provided in a concentration range of between 0.001% to 1%.

22. The method of photodynamic disruption of claim 20 wherein the surface acting agent contains benzalkonium chloride provided in a concentration range of between 0.005% to 0.05%.

23. The method of photodynamic disruption of claim 20 wherein the surface acting agent contains polymyxin B sulfate provided in a concentration range of between 1 to 5 μg/ml.

24. The method of photodynamic disruption of claim 20 wherein the step of providing the surface acting agent precedes the step of providing the photosensitive material by 1 to 60 minutes.

25. The method of photodynamic disruption of claim 1 wherein the step of providing a photosensitive material to the target cell site includes multiple fractionated administrations of photosensitive material to the target cell site.

26. A method of photodynamic disruption comprising:
identifying a target cell site;
providing a photosensitive material to the target cell site;
during a light interval spanning a first period of time, illuminating the target cell site with light from a light source having wavelengths between 450 nm to 900 nm, said light source being repeatedly pulsed ON and OFF at a first duty cycle to provide a periodic light dose to the organism site during the first period of time of between 0.001 J/cm² to 10 J/cm²;
during a dark interval, substantially reducing light delivered to the target cell site by the light source for a second period of time of more than 100 seconds; and
repeating the light interval and then the dark interval during a treatment session to define a second duty cycle, and illuminating the target cell site at the first and second duty cycles during a treatment session to provide a cumulative light dose to the target cell site of between 2 to 400 J/cm² to cause target cell disruption.

27. The method of photodynamic disruption of claim 26 wherein the target cells are associated with one or more of a sterilization procedure, a biofilm eradication procedure, a sterilization procedure of a medical prosthesis, a treatment of an infection at a tissue site, eradication of cancer cells and a fluid or food decontamination process.

28. The method of photodynamic disruption of claim 26 wherein the target cells include microbes, spores, fungi, cancer cells, viruses, prions or plasmids.

29. The method of photodynamic disruption of claim 26 further comprising the step of providing a surface acting agent in association with the target cells.

30. The method of photodynamic disruption of claim 29 wherein the surface acting agent is benzalkonium chloride or polymyxin B sulfate or both.
